(19) United States
(12) Patent Application Publication
(10) Pub. No.: US 2026/0069870 A1
(43) Pub. Date: Mar. 12, 2026

(54) ADJUSTABLE ROTATABLE LEAD CONNECTORS

(71) Applicant: Medtronic, Inc., Minneapolis, MN (US)

(72) Inventors: Jiayu Zhao, Shanghai (CN); Xu Zha, Shanghai (CN); Juan Meng, Shanghai (CN); Hongyang Lu, Shanghai (CN); Zhongping Yang, Woodbury, MN (US); Huiqian Ying, Shanghai (CN)

(21) Appl. No.: 19/101,724

(22) PCT Filed: Aug. 10, 2022
(86) PCT No.: PCT/CN2022/111339

§ 371 Date: Feb. 6, 2025

(51) Int. Cl. A61N 1/375; A61N 1/37; H01R 27/00

(52) U.S. Cl. CPC A61N 1/3752; A61N 1/371; H01R 27/00; H01R 2201/12

(57) ABSTRACT

An adjustable rotational connector is configured to establish electrical communication between an implantable medical lead and a cable of an external testing device while allowing rotation of the implantable medical lead relative to the cable. The coupling includes a pin that is electrically conductive. An adjustable socket is positioned within the pin and is configured to receive lead connectors of different sizes. A bearing is configured to facilitate rotation of the pin relative to the cable. An actuatable element surrounds at least a portion of the adjustable socket. The actuatable element is configured to transition between a first and second position relative to the adjustable socket to allow the adjustable socket to receive and secure the lead connector of the implantable medical lead in the adjustable socket.

### TECHNICAL FIELD

This disclosure generally relates to medical devices, and more specifically, testing implantable medical leads during implantation procedures.

### BACKGROUND

Various types of implantable medical leads have been implanted for treating or monitoring one or more conditions of a patient. Such implantable medical leads may be adapted to allow medical devices to monitor or treat conditions or functions relating to heart, muscle, nerve, brain, stomach endocrine organs, or other organs and their related functions. Implantable medical leads include electrodes and/or other elements for physiological sensing and/or therapy delivery. Implantable medical leads allow the sensing/therapy elements to be positioned at one or more target locations for those functions, while the medical devices electrically coupled to those elements via the leads are at different locations.

Implantable medical leads may be implanted at target locations selected to detect a physiological condition of the patient and/or deliver one or more therapies. For example, implantable medical leads may be delivered to locations within an atria or ventricle to sense intrinsic cardiac signals and deliver pacing or antitachyarrhythmia shock therapy from a medical device coupled to the lead. In other examples, implantable medical leads may be tunneled to locations adjacent a spinal cord or other nerves for delivering pain therapy from a medical device coupled to the lead. Implantable medical leads may include anchoring components to secure a distal end of the lead at the target location.

### SUMMARY

Guiding a distal electrode of an implantable medical lead to achieve an adequate depth within tissue (e.g., cardiac tissue of the interventricular septum) may be necessary for effective delivery of a therapy. For instance, in the case of left bundle branch area pacing (LBBAP) or other conduction system pacing (CSP), insufficient depth of the distal electrode may lead to inadequate therapy, while excessive depth may lead to heart wall perforation. In some examples, a clinician may use real-time electrical signals sensed via the distal electrode may to determine a current location of the distal electrode within tissue. Example electrical signals may include pacing impedance, signals indicative of pacing capture, electrogram signals, etc. One or more of these signals may indicate whether a position of the distal electrode is adequate for a given therapy. For example, a real-time electrogram can provide great support in operation safety and convenience during an implantation procedure for CSP, such as LBBAP.

In some cases, the whole implantable medical lead or a portion thereof is rotated during the implantation procedure (e.g., to advance a fixation member comprising the distal electrode into tissue of a patient, thereby fixating the lead to tissue). However, a test device used to collect signals from the distal electrode and its associated cabling may not be configured to rotate with the lead. For example, rotation of the lead may cause the cabling of the test device to become tangled, potentially interfering with the implantation procedure and/or introducing undesirable noise and resistance. Additionally, the test device cabling may not be compatible with different types of lead interfaces (e.g., IS-1, DF-4 and IS-4). This may disadvantageously result in a physician being unable to obtain a real-time electrogram signal depending on the lead being used during an operation, or requiring the physician to acquire and select from amongst different test devices and/or cabling for different lead types.

An adjustable rotational connector according to the present disclosure may include features to maintain an electrical connection between an electrode of an implantable medical lead and a test device during rotation of the lead. In some examples, the adjustable rotational connector may reduce the presence of noise in signals sensed by the distal electrode. These features may allow a clinician to continuously observe real-time electrical signals, or data derived therefrom, during rotation of the lead, which may reduce the time and effort needed to identify an adequate implant position/depth for the distal electrode. Furthermore, the rotational connector according to the present disclosure may be configured to be adjustable to receive lead interfaces of different sizes associated with different types of leads.

In some examples, an adjustable rotational connector is configured to establish electrical communication between an implantable medical lead and a cable of an external testing device, wherein the adjustable rotational connector comprises: a pin that is electrically conductive; an adjustable socket that is electrically conductive and configured to elastically deform to receive a lead connector of the implantable medical lead, wherein a proximal end of the socket is mechanically and electrically coupled to the pin to provide electrical communication between the pin and the lead connector, and wherein the adjustable socket is configured to receive lead connectors of different sizes; a bearing configured to facilitate rotation of the pin relative to the cable; an actuatable element that surrounds at least a portion of the socket and: while in a first position relative to the adjustable socket, be disengaged from the socket to allow the socket to elastically deform to receive the lead connector of the implantable medical lead; and while in a second position relative to the adjustable socket, be engaged to the adjustable socket to secure the lead connector of the implantable medical lead in the socket.

In some examples, a system comprises: an implantable medical lead comprising: a distal electrode; and a lead connector; a cable in electrical communication with an external test device; an adjustable rotational connector configured to establish electrical communication between the implantable medical lead and the cable, the adjustable rotational connector comprising: a pin that is electrically conductive; an adjustable socket that is electrically conductive and configured to elastically deform to receive the lead connector of the implantable medical lead, wherein a proximal end of the socket is mechanically and electrically coupled to the pin to provide electrical communication between the pin and the lead connector, and wherein the adjustable socket is configured to receive lead connectors of different sizes; a bearing configured to facilitate rotation of the pin relative to the cable; an actuatable element that surrounds at least a portion of the socket and: while in a first position relative to the adjustable socket, be disengaged from the socket to allow the socket to elastically deform to receive the lead connector of the implantable medical lead; and while in a second position relative to the adjustable socket, be engaged to the adjustable socket to secure the lead connector of the implantable medical lead in the socket.

In some examples, a method comprises: surrounding, by an actuatable element, at least a portion of an adjustable socket of an adjustable rotational connector, wherein the adjustable rotational connector is configured to establish electrical communication between an implantable medical lead and a cable of an external testing device, wherein the adjustable socket is electrically conductive and configured to elastically deform to receive a lead connector of the implantable medical lead, wherein a proximal end of the socket is mechanically and electrically coupled to a pin of the adjustable rotational connector to provide electrical communication between the pin and the lead connector, and wherein the adjustable socket is configured to receive lead connectors of different sizes; while transitioning from a second position relative to the adjustable socket to a first position relative to the adjustable socket, mechanically disengaging, by the actuatable element, the adjustable socket to allow the adjustable socket to elastically deform to receive the lead connector of the implantable medical lead; while transitioning from the second position relative to the adjustable socket to the first position relative to the adjustable socket, mechanically engaging, by the actuatable element, the adjustable socket to secure the lead connector of the implantable medical lead in the socket; and rotating, by a bearing of the adjustable rotational connector, the pin relative to the cable.

In some examples, an adjustable rotational connector configured to electrically couple an implantable medical lead and an external testing device while permitting axial rotation of the lead relative to the external testing device, wherein the adjustable rotational connector comprises: a stator configured to be connected to the external testing device by a cable, the stator comprising an inner recess; a rotor received within the inner recess of the stator; and at least one bearing within the inner recess of the stator, the at least one bearing configured to facilitate relative rotation between the stator and the rotor along a longitudinal axis, wherein the rotor comprises: an adjustable socket that is electrically conductive and configured to elastically deform to receive a lead connector of the implantable medical lead, wherein the socket is electrically coupled to the cable, and wherein the adjustable socket is configured to receive lead connectors of different sizes; and an actuatable element that surrounds at least a portion of the socket and move along the longitudinal axis to transition between a first position and a second position relative to the socket, wherein: while in a first position relative to the adjustable socket, be disengaged from the socket to allow the socket to elastically deform to receive the lead connector of the implantable medical lead; and while in a second position relative to the adjustable socket, be engaged to the adjustable socket to the lead connector of the implantable medical lead in the socket.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example medical device system in accordance with techniques of this disclosure.

FIG. 2 is a conceptual diagram illustrating an example system for testing depth of a distal electrode of an implantable medical lead in accordance with techniques of this disclosure.

FIG. 3A is a cross-sectional diagram illustrating an example adjustable rotational connector in accordance with techniques of this disclosure.

FIG. 3B is an exploded diagram of the adjustable rotational connector of FIG. 3A, the adjustable rotational connector including a stator and a rotor in accordance with techniques of this disclosure.

FIG. 3C is an exploded diagram of the stator of FIG. 3B in accordance with techniques of this disclosure.

FIG. 3D is an exploded diagram of the rotor of FIG. 3C in accordance with techniques of this disclosure.

FIG. 4A is a cross-sectional diagram illustrating a portion of an example adjustable rotational connector with an increased inner diameter in accordance with techniques of this disclosure.

FIG. 4B is a cross-sectional diagram illustrating a portion of an example adjustable rotational connector with a decreased inner diameter in accordance with techniques of this disclosure.

FIG. 5A is a cross-sectional diagram illustrating a portion of an example adjustable rotational connector and an example lead connector in accordance with techniques of this disclosure.

FIG. 5B is a cross-sectional diagram illustrating a portion of an example adjustable rotational connector and an example lead connector in accordance with techniques of this disclosure.

FIG. 6 is a flow diagram illustrating an example technique for coupling a lead to an adjustable rotational connector.

FIG. 7 is a flow diagram illustrating an example technique for testing an implantable medical lead during an implantation procedure using an adjustable rotational connector.

### DETAILED DESCRIPTION

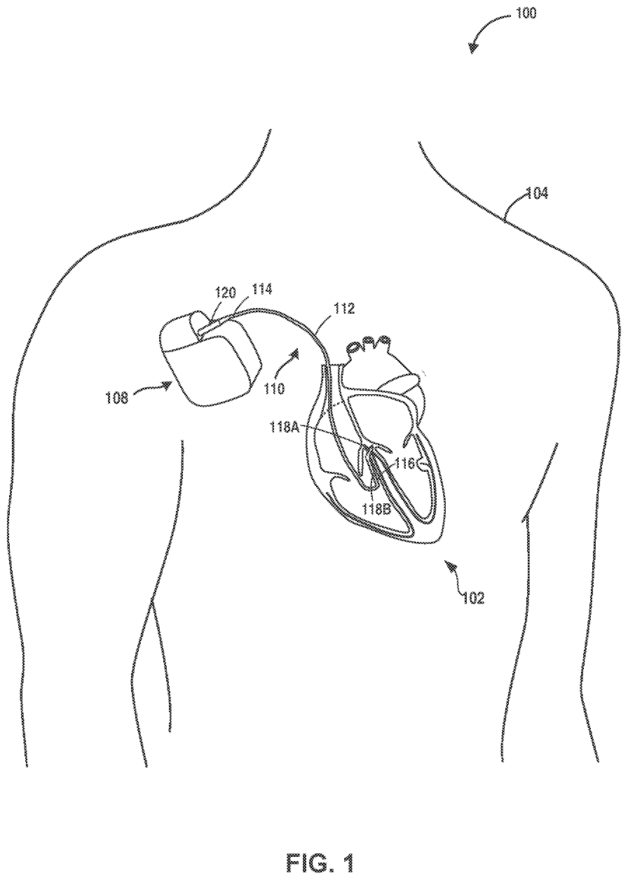

FIG. 1 is a conceptual diagram illustrating an example medical device system 100 (“system 100”) for delivering CSP, such as LBBAP, to a heart 102 of a patient 104. As illustrated by example system 100 in FIG. 1, system 100 may include an implantable medical device 108 (“IMD 108”) with cardiac pacing capabilities. IMD 108 is connected to an implantable medical lead 110 (“lead 110”) that includes a lead body 112 extending from a proximal portion 114 of lead 110 (“lead proximal portion 114”) to a distal portion 116 of lead 110 (“lead distal portion 116”). Lead proximal portion 114 may be operably coupled to IMD 108. Although primarily described herein with respect to LBBAP, the techniques of this disclosure may be applied to other regions of the heart, including other portions of the conduction system of the heart, such as the His-Purkinje conduction system (HPCS) or the right bundle branch (RBB). Furthermore, although primarily described herein in the context of cardiac pacing, the techniques of this disclosure may be applied to non-cardiac contexts, such as neurostimulation.

IMD 108 may sense electrical signals attendant to the depolarization and repolarization of heart 102, e.g., a cardiac EGM, via electrodes on lead 110 and/or the housing of IMD 108. IMD 108 may also deliver therapy in the form of electrical signals, e.g., cardiac pacing, to heart 102 via electrodes located on lead 110. In the illustrated example, lead 110 includes a distal electrode 118A at lead distal portion 116 and a proximal electrode 118B located proximally of distal electrode 118A (collectively, “electrodes 118”). In other examples, lead 110 may include more or fewer electrodes 118, such as examples in which lead 110 includes only electrode 118A.

Lead 110 may extend into heart 102 of patient 104 to sense electrical activity of heart 102 and/or deliver electrical stimulation to heart 102. In the example shown in FIG. 1, lead 110 extends through one or more veins (not shown), the superior vena cava (not shown), the right atrium, and into cardiac tissue of the interventricular septum. System 100 may include additional leads coupled to IMD 108, such as a left ventricular (LV) lead that extends through one or more veins, the vena cava, the right atrium, and into the coronary sinus to a region adjacent to the free wall of the left ventricle of heart 102, and/or a lead that extends into the right atrium.

Lead 110, e.g., distal electrode 118A, may be positioned to provide pacing to the LBB. Providing pacing to the LBB is sometimes referred to as “LBBAP” or “LBBP.” In the illustrated example, distal electrode 118A is positioned within cardiac tissue of the interventricular septum. In other examples, lead 110 and distal electrode 118A may be implanted at positions to provide pacing to other portions of heart 102, such as the HPCS or RBB.

In some examples, distal electrode 118A may be carried by a distal end of lead 110. In addition to being electrically active, distal electrode 118A may be configured to grasp tissues at or near a target site and substantially secure a distal end of lead 110 to the target site. In other words, distal electrode 118A may be configured to substantially maintain an orientation of lead 110 with respect to the target site by penetrating tissue. That is, in some examples, distal electrode 118A may be, include, or be included in a fixation member. For instance, distal electrode 118A may be an uninsulated portion of a fixation member. In some examples, distal electrode 118A may include one or more fixation tines of any shape, including, but not limited to, helically shaped fixation tines. For example, distal electrode 118A may take the form of a fixed helix, a tine tip electrode, etc.

Electrode 118B may take the form of a ring electrode electrically insulated from electrode 118A. In some examples, distal electrode 118A may be positioned within the cardiac tissue such that pacing stimulation delivered via distal electrode 118A activates the LBB. During an implantation procedure for lead 110, an implanting physician may position a distal end of lead 110 at a desired location, and fix distal electrode 118A distally from the distal end of lead 110 to a desired depth within the cardiac tissue, e.g., the interventricular septum.

Lead 110 may include a lead electrical connector 120 (sometimes referred to herein as “connector 120” or “lead connector 120”), such as an IS-1 connector, DF-4 connector, or IS-4 connector, configured to establish electrical communication between IMD 108 and electrodes 118. Connector 120 may be configured to electrically communicate with circuitry of IMD 108. Connector 120 includes one or more conductors (not shown) configured to electrically communicate with electrodes 118. In some examples, each of electrodes 118A and 118B is electrically coupled to a respective conductor within connector 120 and thereby coupled to circuitry within IMD 108. In examples, connector 120 is configured such that electrical communication with distal electrode 118A and proximal electrode 118B occurs substantially independently to, e.g., facilitate correct placement of electrodes 118 and/or obtain a better electrical signal (lower threshold, lower impedance, etc.).

As described in greater detail below, placement of CSP leads may cause particular challenges to ensure proper location and depth of electrode 118A (e.g., rotations of a lead including a distal helix) to capture a portion of the CS. Conventional alligator clips used to electrically connect leads to a test device during implantation to test electrical adequacy of implantation depth may similarly cause challenges and delay the process for implantation involving lead rotation. Further, different commercial leads may have different connectors, which in turn may have different sizes. As a result, some commercial leads may be incompatible with certain testing systems.

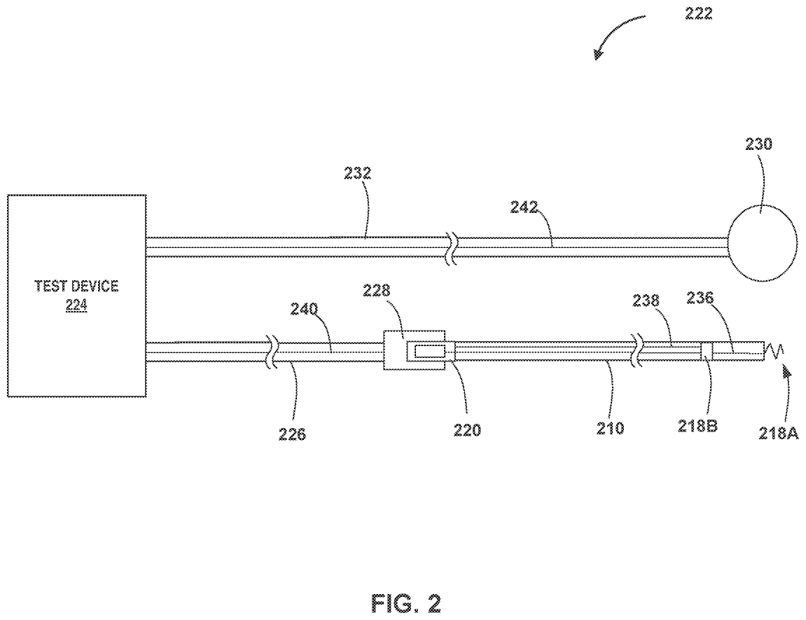

FIG. 2 is a conceptual diagram illustrating an example system 222 for testing an implantable medical lead 210 (“lead 210”) during an implantation procedure. Lead 210 may be substantially similar to lead 110 shown in FIG. 1. As illustrated by the example of FIG. 2, system 222 includes a test device 224 that is connected to lead 210 via a first cable 226. More particularly, first cable 226 includes an adjustable rotational connector 228 configured to selectively physically and electrically connect first cable 226 to a lead connector 220. System 222 also includes an auxiliary electrode 230, which may be attached externally to a patient, e.g., via an adhesive patch. Auxiliary electrode 230 may be connected to test device 224 via a second cable 232.

Adjustable rotational connector 228 may be configured such that lead 210 may rotate relative to first cable 226 while maintaining an electrical connection between test device 224 and a distal electrode 218A. For example, adjustable rotational connector 228 may include a rotational coupling (e.g., a rotor). Adjustable rotational connector 228 may be configured to electrically connect to distal electrode 218A and test device 224. For example, adjustable rotational connector 228 may be configured to conduct a cardiac electrogram signal in which left bundle branch tissue features can be detected during rotation of lead 210. In another example, adjustable rotational connector 228 may be configured to conduct a cardiac electrogram signal in which His-Purkinje conduction system features can be detected during rotation of lead 210.

Lead 210 may include a first conductor 236 electrically connected to distal electrode 218A and a second conductor 238 electrically connected to a proximal electrode 218B. First conductor 236 may be electrically connected to adjustable rotational connector 228. First cable 226 includes a third conductor 240 which is electrically connected or connectable to both adjustable rotational connector 228 and lead connector 220 vian adjustable rotational connector 228, and test device 224. When lead 210, adjustable rotational connector 228, first cable 226, and test device 224 are connected, first conductor 236 and third conductor 240 electrically connect distal electrode 218A to test device 224. Auxiliary electrode 230 is also connected or connectable to test device 224 via a conductor 242 of second cable 232. In the illustrated example, adjustable rotational connector 228 via first cable 226 is configured to electrically connect one electrode of lead 210, distal electrode 218A, to test device 224 in a unipolar configuration. In other examples, adjustable rotational connector 228 and test device 224 may be connected to both electrodes 218 of lead 210 in a bipolar configuration, and auxiliary electrode 230 and second cable 232 may be omitted.

Test device 224 receives one or more signals sensed using distal electrode 218A with auxiliary electrode 230 acting as a reference electrode. In some examples, test device 224 measures a pacing impedance signal using distal electrode 218A. In some examples, test device 224 receives a cardiac EGM signal sensed using distal electrode 218A with auxiliary electrode 230 acting as a reference electrode. An implanting physician may use the signals and/or values derived from the signals to determine whether a current position/depth of distal electrode 218A is adequate for sensing and therapy delivery by IMD 108 via distal electrode 218A. In the case of LBBAP pacing, for example, the presence of LBB features in the cardiac EGM (e.g., features indicative of the electrical activity of the LBB) may indicate an adequate position/depth of distal electrode 218A.

In some cases, lead 210 (or a portion thereof) is rotated during the implantation procedure (e.g., to fixate lead 210). However, test device 224, first cable 226, and second cable 232 may not be able to rotate with lead 210 (e.g., without becoming entangled). Furthermore, rotation of lead 210 may introduce artifacts or other noise into the signals used to determine whether its position/depth is adequate. For example, relative rotation of portions of the conductive pathway may introduce noise, e.g., due to make/break events occurring during the relative rotation. The noise may corrupt the signals such that the adequacy of the position/depth of electrode 218A cannot be determined during rotation. In accordance with techniques of this disclosure, adjustable rotational connector 228 may allow relative rotation of lead 210 and first cable 226, and mitigate noise associated with such rotation.

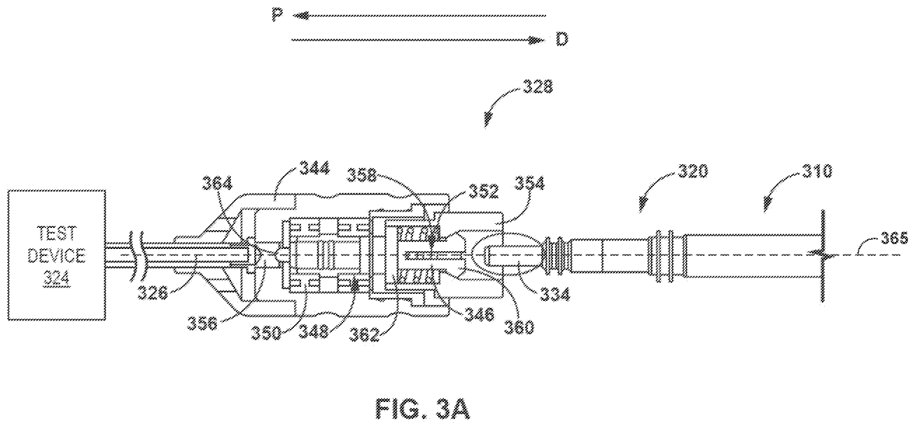

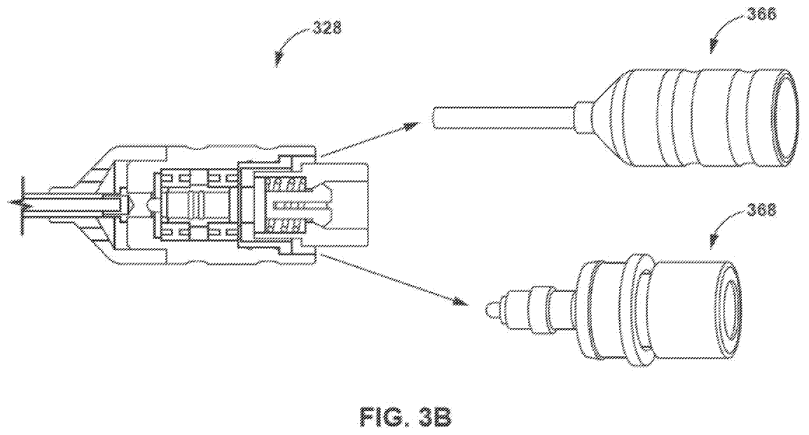

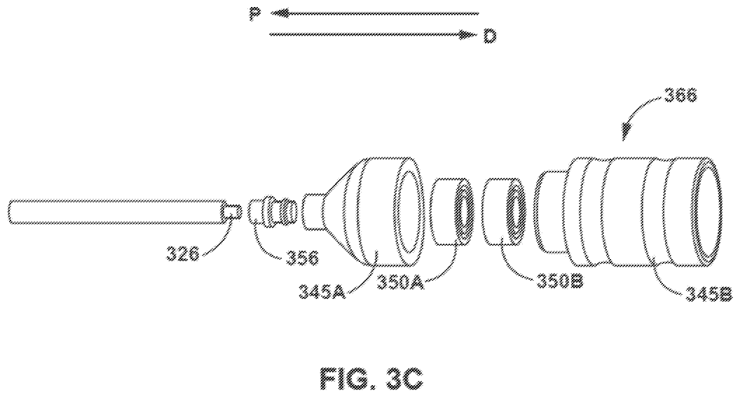

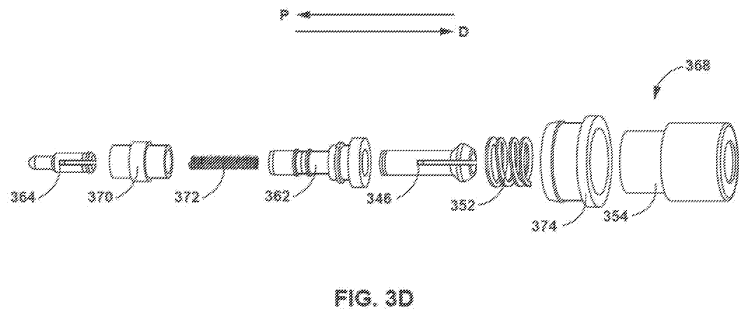

FIG. 3A is a cross-sectional diagram illustrating an example adjustable rotational connector 328. adjustable rotational connector 328 may be substantially similar to adjustable rotational connector 228 shown in FIG. 2. In accordance with techniques of this disclosure, rotational electrical adjustable rotational connector 328 may not only establish electrical communication between an implantable medical lead 310 (“lead 310”) and a test device 324, but also facilitate rotation between lead 310 and test device 324. Lead 310 may be substantially similar to lead 110 shown in FIG. 1 and/or lead 210 shown in FIG. 2, and test device 324 may be substantially similar to test device 224. Additionally, in accordance with techniques of this disclosure, adjustable rotational connector 328 may be configured to couple to a variety of lead connector types, including but not limited to IS-1 connectors, DF-4 connectors, and IS-4 connectors. As a result, adjustable rotational connector 328 may achieve compatibility with different sizes of pacing leads and electrical transmission while rotating with little resistance.

As shown in FIG. 3A, adjustable rotational connector 328 includes a connector body 344. Connector body 344 may be formed from polyether ether ketone (PEEK) or other insulative materials. Connector body 344 may be configured to at least partially house (e.g., mechanically support, contain, etc.) an adjustable lead connector socket 346 (“socket 346”), a pin 348, one or more bearings 350 (“bearing 350”), which may be insulative or conductive, an elastically deformable member 352, an actuatable element 354, and an electrical contact structure 356 (“contact structure 356”).

Socket 346 may be configured to receive a proximal end 334 of a lead connector 320, in this way establishing electrical communication with one or more electrodes of lead 310, e.g., the distal electrode of the lead. Socket 346 may be electrically conductive. Socket 346 may be formed from conductive materials, such as copper, silver, gold, etc. Socket 346 may be configured to elastically deform (e.g., in response to constriction). For example, socket 346 may have a structure amenable to constriction by one or more applied forces. In some examples, and as shown in FIG. 3, socket 346 may define one or more indentations 358 (e.g., notch, slot, slit, channel, etc.) that allows socket 346 to elastically deform to receive lead connector 320. For example, socket 346 may define one or more slits extending axially along socket 346 from a distal end of socket 346 such that a distal portion 360 of adjustable socket 346 is radially elastically deformable. Socket 346 may resume a normal shape, such as a not constricted shape, after being elastically deformed in accordance with techniques of this disclosure.

Actuatable element 354 of adjustable rotational connector 328 may surround at least a portion of socket 346. For instance, actuatable element 354 may define a channel in which socket 346 and elastically deformable member 352 are at least partially positioned. Actuatable element 354 may be configured to engage and disengage socket 346. The engagement of actuatable element 354 to socket 346 may be mechanical, electrical, etc. disengagement of actuatable element 354 to socket 346 may be mechanical, electrical, etc. In some examples, actuatable element 354 may be mechanically or electrically moved, rotated, pulled, pressed, etc., to engage and disengage socket 346. For example, a clinician may mechanically move, rotate, pull, press, etc., actuatable element 354 to engage and disengage socket 346.

Actuatable element 354 may be configured to, while in a first position relative to socket 346, be disengaged from socket 346 to allow socket 346 to receive lead connector 320. For example, a clinician may move actuatable element 354 to the first position by compressing elastically deformable member 352 in a proximal direction (indicated by the “P” arrow). While in the first position, actuatable element 354 may be disengaged from distal portion 360 of socket 346, allowing an inner diameter of at least distal portion 360 of socket 346 to increase. While actuatable element 352 is in the first position, socket 346 may be configured to receive a lead connector of an IS-1 connector, a DF-4 connector, and an IS-4 lead connector size configuration. In this way, socket 346 may be configured to receive lead connectors of different sizes, such as lead connectors with inner diameters between about 0.5 millimeters (mm) and about 3 mm and lengths between about 3 mm and 8 mm.

Socket 346 may be configured to secure lead connector 320. For example, once lead connector 320 is positioned within socket 346, actuatable element 354 may be configured to, while in a second position relative to socket 346, be engaged to socket 346 to elastically deform socket 346 to secure lead connector 320 in socket 346. In some examples, elastically deformable member 352 may be configured to provide a biasing force to move actuatable element 354 to the second position. For instance, a clinician may release actuatable element 354. As a result, elastically deformable member 352 may expand in a distal direction (indicated by the “D” arrow) such that actuatable element 352 engages (e.g., contacts) distal portion 360 of socket 346. The contact between actuatable element 352 and socket 346 may cause socket 346 to radially compress onto lead connector 320. As such, socket 346 may clamp down onto at least a portion of lead connector 320, securing lead connector 320 within socket 346 and ensuring electrical contact therebetween.

Pin 348 may be configured to couple to socket 346. For example, a proximal end of socket 346 may be mechanically and electrically coupled to pin 348 to provide electrical communication between pin 348 and lead connector 320. In some examples, pin 348 may include a pin base 362 (“base 362”) and a pin tip 364 (“tip 364”). Base 362 may define a recess. At least a segment of socket 346 may be positioned or otherwise disposed within the recess of pin 348. Tip 364 may be in contact with contact structure 356. Pin 348 may be electrically conductive. For example, both base 362 and tip 364 may be electrically conductive. Base 362 and tip 364 may be formed from conductive materials.

Bearing 350 may be configured to facilitate rotation of pin 348 (and thus socket 346 and lead 310) relative to first cable 326. For example, an inner rotating component of bearing 350 may be secured to pin 348, while an outer rotating component of bearing 350 may be secured to connector body 344. Bearing 350 may be configured such that the inner rotating component of bearing 350 is able to freely rotate (e.g., with little resistance) relative to the outer rotating component.

Contact structure 356 may be configured to establish electrical communication between pin 348 and first cable 326. Contact structure 356 may be formed from conductive materials. As shown in FIG. 3, first cable 326 is in electrical contact with contact structure 356. As further shown in FIG. 3, tip 364 of pin 348 is in electrical contact with contact structure 356. Pin 348 may be rotatable along a longitudinal axis 365 relative to contact structure 356.

In some examples, pin 348 may include a pin bearing (e.g., an elastically deformable member, such as a spring). The pin bearing may be configured to achieve stable signal transmission from lead connector 320 to first cable 326 (e.g., by reducing the degree of axial movement of tip 364 of pin 348 during rotation of lead 310). In this way, adjustable rotational connector 328 may mitigate noise that could otherwise be introduced into the signals received by test device 324.

FIG. 3B is an exploded diagram of adjustable rotational connector 328 shown in FIG. 3A. As shown in FIG. 3B, adjustable rotational connector 328 includes a stator 366 and a rotor 368. Stator 366 may be configured to be rotationally fixed relative to test device 324. For example, when lead 310 is being rotated, stator 366 may not rotate with lead 310 but instead remain substantially stationary. Rotor 368 may be configured to be rotationally fixed relative to lead 310 and to rotate relative to stator 366. For example, when lead 310 is being rotated, rotor 368 may rotate with lead 310 (such that rotor 368 rotates relative to stator 366 and in turn test device 324).

FIG. 3C is an exploded diagram of stator 366. As shown in FIG. 3C, stator 366 includes a variety of components. For example, stator 366 may include first cable 326, contact structure 356, a proximal connector body component 345A, a distal connector body component 345B (collectively, “connector body components 345”), a first insulative bearing 350A, and a second insulative bearing 350B (collectively, “bearing 350”). Connector body 344, which includes connector body components 345, may be configured to house the various components of stator 366. Stator 366 may be configured to be rotationally fixed relative to test device 324. For example, when lead 310 is being rotated, stator 366 may not rotate with lead 310 but instead remain substantially stationary.

FIG. 3D is an exploded diagram of rotor 368. As shown in FIG. 3D, rotor 368 includes a variety of components. For example, stator 368 may include tip 364, an insulating support 370, a pin biasing member 372 (“biasing member 372”), base 362, socket 346, elastically deformable member 352, an actuatable element assembly 374, and actuatable element 354. Insulating support 370 may be configured to house at least a portion of tip 364, biasing member 372, and base 362. Actuatable element assembly 374 may be configured to house at least a portion of actuatable element 354.

Pin 348 may include base 362 and tip 364. Pin 348 may be configured to rotate relative to contact structure 356. For example, pin 348 may be positioned within and operatively coupled to bearing 350. In this way, pin 348 may rotate with an implantable medical lead while contact structure 356 remains stationary. Additionally, tip 364 of pin 348 may be in electrical contact with contact structure 356.

In some examples, pin 348 may include biasing member 372 (e.g., an elastically deformable member, such as a spring). Biasing member 372 may be configured to force tip 364 against contact structure 356. By reducing axial movement of tip 364 in this way, biasing member 372 may help achieve stable signal transmission during rotation of lead 310. Thus, adjustable rotational connector 328 may mitigate noise that could otherwise be introduced into the signals received by a test device.

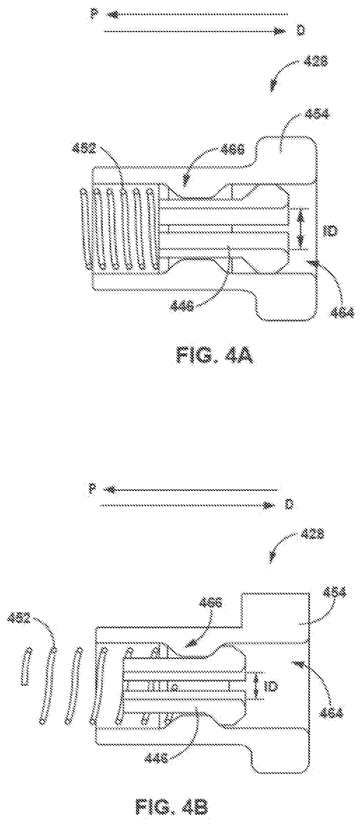

FIGS. 4A-4B are cross-sectional diagrams illustrating a portion of an example adjustable rotational connector 428. Adjustable rotational connector 428 may be substantially similar to adjustable rotational connector 228 shown in FIG. 2 and/or adjustable rotational connector 328 shown in FIG. 3. As shown in FIGS. 4A-4B, adjustable rotational connector 428 includes a socket 446, an elastically deformable member 452, and an actuatable element 454. FIG. 4A shows actuatable element 454 in a first position relative to socket 446. While actuatable element 454 is in the first position, adjustable rotational connector 428 may be configured to receive a lead connector (e.g., lead connector 320). FIG. 4B shows actuatable element 454 in a second position relative to socket 446. While actuatable element 454 is in the second position, adjustable rotational connector 428 may be configured to secure the lead connector.

As noted above, adjustable rotational connector 428 may be compatible with a variety of lead connector sizes. For example, socket 446 may be elastically deformable such that an inner diameter of socket 446 (indicated by “ID”) may be adjustable, allowing for insertion and engagement of a lead connector (e.g., lead connector 320) within socket 446. In some examples, actuatable element 454 may be configured to interact with socket 446 and elastically deformable member 452 to facilitate the change in the inner diameter of socket 446.

Socket 446 and elastically deformable member 452 may be at least partially positioned within a channel 464 of actuatable element 454. In some examples, a portion 466 of channel 464 may be dimensioned such that the wall defining portion 466 is engaged to (e.g., in contact with) socket 446 to radially compress socket 446 onto a lead connector (e.g., lead connector 320) while actuatable element 454 is in the second position relative to socket 446. As shown in FIGS. 4A-4B, the wall may be tapered such that the movement of pressing member 454 distally relative to socket 446 causes a radial constriction of socket 446.

In some examples, at least a section of portion 466 may have an inner diameter that is less than an inner diameter of channel 464 immediately distal of that section of portion 466 such that the wall is disengaged from socket 446 while actuatable element 454 is in the first position relative to socket 446. Actuatable element 454 may transition from the second position relative to socket 446 to the first position relative to socket 446 in response to a clinician moving actuatable element 454 in a proximal direction (indicated by the “P” arrow). As shown in FIG. 4A, when actuatable element 454 is in the first position, actuatable element 454 no longer radially constricts socket 446, allowing the inner diameter of socket 446 to increase. When in this increased inner diameter configuration, socket 446 may have an inner diameter sized to receive a variety of lead connector sizes.

While actuatable element 454 is in the first position, elastically deformable member 452 may be compressed and providing a biasing force to move actuatable element 454 to the second position. For example, a clinician may release actuatable element 454 (e.g., after inserting lead connector 320 into socket 446), and elastically deformable member 452 may in turn expand in a distal direction (indicated by the “D” arrow) such that the wall defining portion 466 is engaged to socket 446 to radially compress socket 446 onto a lead connector, securing the lead connector within socket 446.

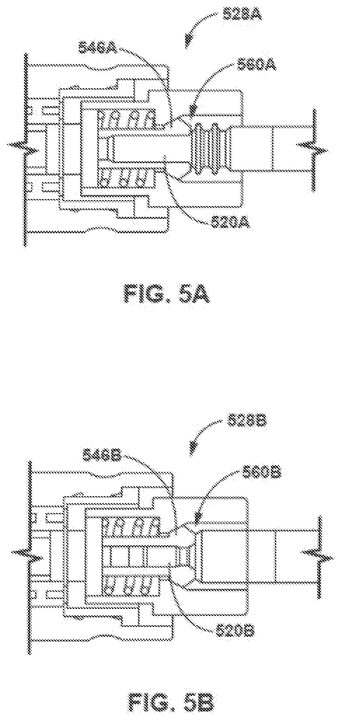

FIG. 5A is a cross-sectional diagram illustrating a portion of an example adjustable rotational connector 528A and a lead connector 520A. FIG. 5B is a cross-sectional diagram illustrating a portion of an example adjustable rotational connector 528B and a lead connector 520B. Adjustable rotational connectors 528A-528B (collectively, “adjustable rotational connectors 528”) are substantially similar to adjustable rotational connector 228 shown in FIG. 2, adjustable rotational connector 328 shown in FIG. 3, and/or adjustable rotational connector 428 shown in FIG. 4. Lead connector 520A and lead connector 520B (collectively, “lead connectors 520”) are substantially similar to lead connector 220 shown in FIG. 2 and/or lead connector 320 shown in FIG. 3.

In general, adjustable rotational connectors in accordance with techniques of this disclosure (e.g., adjustable rotational connectors 528) may be configured to be compatible with a variety of lead connectors (e.g., lead connectors 520). That said, in some examples, sockets may be customized to improve engagement with lead connectors. For example, FIG. 5A illustrates a socket 546A having a distal portion 560A with an inner diameter that is uniform. The uniform inner diameter of distal portion 560A may allow for greater purchase of lead connector 520A, which is illustrated in FIG. 5A as having a flat outer surface. In another example, FIG. 5B illustrates a socket 546B having a distal portion 560B with one or more features extending from the inner surface of distal portion 560B. These features may be configured to contact lead connector 520B when lead connector 520B is secured by socket 546B.

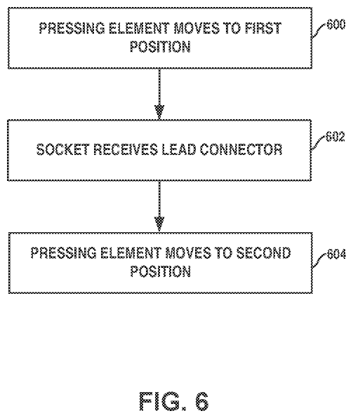

FIG. 6 is a flow diagram illustrating an example technique for coupling a lead to an adjustable rotational connector. The techniques of FIG. 6 are described with respect to adjustable rotational connector 328 and lead 310. However, the techniques of FIG. 6 may be applied to any device or combination of devices described herein.

Actuatable element 354 may move to a first position relative to socket 346 (600). For example, a clinician may move actuatable element 354 to the first position by compressing elastically deformable member 352 in a proximal direction (indicated by the “P” arrow). While in the first position, actuatable element 354 may be disengaged from distal portion 360 of socket 346, allowing an inner diameter of at least distal portion 360 of socket 346 to increase.

While actuatable element 352 is in the first position, socket 346 may receive lead connector 320 (602). Lead connector 320 may be, for example, a DF-4 connector, or an IS-4 lead connector.

Socket 346 may move to a second position relative to socket 346 (604). For example, elastically deformable member 352 may provide a biasing force to move actuatable element 354 to the second position such that actuatable element 352 engages (e.g., contacts) distal portion 360 of socket 346. The contact between actuatable element 352 and socket 346 may cause socket 346 to radially compress onto lead connector 320. As such, socket 346 may clamp down onto at least a portion of lead connector 320, securing lead connector 320 within socket 346 and ensuring electrical contact therebetween.

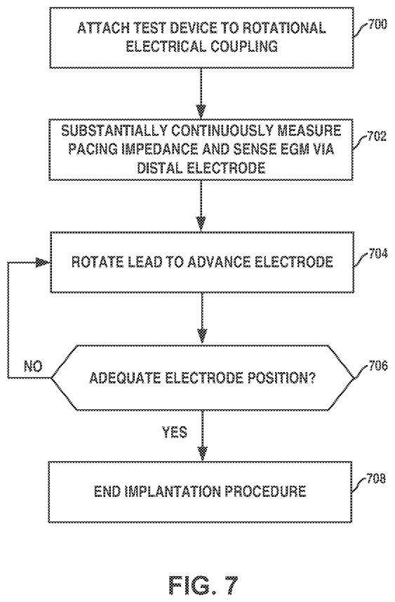

FIG. 7 is a flow diagram illustrating an example technique for a testing lead during an implantation procedure using an adjustable rotational connector. The techniques of FIG. 7 are described with system 222. However, the techniques of FIG. 7 may be applied to any device or combination of devices described herein.

Test device 224 may be electrically connected to rotational electrical adjustable rotational connector 328 (700). In some examples, test device 224 comprises adjustable rotational connector 228 or other connector configured to engage a portion of rotational electrical adjustable rotational connector 228, such as first conductive component 60A. In this way, distal electrode 218A of lead 210 is electrically connected to test device 224 via rotational electrical adjustable rotational connector 228 as described herein.

Distal electrode 218A of lead 210 may be positioned adjacent to cardiac tissue, e.g., the interventricular septum, at a desired location for sensing and delivery of therapy, e.g., for LBBAP. With distal electrode 218A electrically connected to test device 224 and located as desired relative to the cardiac tissue, test device 224 may begin to measure impedance and sense a cardiac EGM via distal electrode 218A (702). Once initiated, the measurement and sensing by test device 224 may be substantially continuous, e.g., at a sampling rate during a period of time that includes a plurality of cardiac cycles and a plurality of positions/depths of distal electrode 218A. While test device 224 measures or senses one or more signals, lead 210 or a portion thereof may be rotated to advance distal electrode 218A within cardiac tissue (704). In some cases, distal electrode 218A may additionally be repositioned to different entry points of and trajectories through cardiac tissue.

Based on an output of test device 224 that is based on the one or more signals obtained via distal electrode 218A, the implanting physician may determine whether a current position/depth of distal electrode 218A is adequate for the sensing and delivery of therapy (706). If the current position/depth is not adequate (NO of 706), then the physician may continue to rotate lead 210 relative to heart tissue while test device 224 continues to acquire one or more signals via distal electrode 218A. If the current position/depth is adequate (YES of 706), then the physician may end that portion of an implantation procedure and lead 210 (708).

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. An adjustable rotational connector configured to establish electrical communication between an implantable medical lead and a cable of an external testing device, wherein the adjustable rotational connector comprises:
a pin that is electrically conductive;
an adjustable socket that is electrically conductive and configured to elastically deform to receive a lead connector of the implantable medical lead, wherein a proximal end of the adjustable socket is mechanically and electrically coupled to the pin to provide electrical communication between the pin and the lead connector, and wherein the adjustable socket is configured to receive lead connectors of different sizes;
a bearing configured to facilitate rotation of the pin relative to the cable; and
an actuatable element that surrounds at least a portion of the adjustable socket, wherein the actuatable element is configured to:
while in a first position relative to the adjustable socket, be disengaged from the adjustable socket to allow the adjustable socket to elastically deform to receive the lead connector of the implantable medical lead; and
while in a second position relative to the adjustable socket, be engaged to the adjustable socket to secure the lead connector of the implantable medical lead in the adjustable socket.

2. The adjustable rotational connector of claim 1, further comprising an electrical contact structure configured to establish electrical communication between the pin and the cable, wherein the pin is rotatable along a longitudinal axis relative to the contact surface, and wherein the pin comprises:
a pin base defining a recess;
a pin tip; and
a pin biasing member configured to force the pin tip against the contact surface.

3. The adjustable rotational connector of claim 1. wherein the adjustable socket is configured to receive a lead connector of an IS-1 connector, a DF-4 connector, and an IS-4 lead connector size configuration.

4. The adjustable rotational connector of any of claim 1, wherein the actuatable element defines a channel in which the adjustable socket and an elastically deformable member are at least partially positioned, wherein the elastically deformable member is configured to provide a biasing force to move the actuatable element to the second position, and wherein a portion of the channel is dimensioned such that a wall defining the portion of the channel is engaged to the adjustable socket to radially compress the adjustable socket onto the lead connector while the actuatable element is in the second position relative to the adjustable socket.

5. The adjustable rotational connector of claim 4, wherein the elastically deformable member is a spring.

6. The adjustable rotational connector of claim 4, wherein at least a section of the portion of the channel has an inner diameter that is less than an inner diameter of the channel immediately distal of the section such that the wall is disengaged from the adjustable socket while the actuatable element is in the first position relative to the adjustable socket.

7. The adjustable rotational connector of claim 1, wherein an inner diameter of a distal portion of the adjustable socket is uniform.

8. The adjustable rotational connector of claim 1, wherein the adjustable socket further comprises one or more features that extend from an inner surface of the adjustable socket, and wherein the one or more features are configured to contact the lead connector when the lead connector is secured by the adjustable socket.

9. The adjustable rotational connector claim 1, wherein the adjustable socket defines define one or more indentations that allows the adjustable socket to elastically deform to receive the lead connector.

10. The adjustable rotational connector of claim 9, wherein the one or more indentations comprise one or more slits extending axially along the adjustable socket from a distal end of the adjustable socket such that a distal portion of the adjustable socket is radially elastically deformable.

11. A system comprising:
an implantable medical lead comprising:
a distal electrode; and
a lead connector;
a cable in electrical communication with an external test device; and
an adjustable rotational connector configured to establish electrical communication between the implantable medical lead and the cable, the adjustable rotational connector comprising:
a pin that is electrically conductive;
an adjustable socket that is electrically conductive and configured to elastically deform to receive the lead connector of the implantable medical lead, wherein a proximal end of the socket is mechanically and electrically coupled to the pin to provide electrical communication between the pin and the lead connector, and wherein the adjustable socket is configured to receive lead connectors of different sizes;
a bearing configured to facilitate rotation of the pin relative to the cable; and
an actuatable element that surrounds at least a portion of the adjustable socket,
wherein the actuatable element is configured to:
while in a first position relative to the adjustable socket, be disengaged from the socket to allow the socket to elastically deform to receive the lead connector of the implantable medical lead; and
while in a second position relative to the adjustable socket, be engaged to the adjustable socket to secure the lead connector of the implantable medical lead in the socket.

12. The system of claim 11, wherein the adjustable rotational connector is configured to conduct a cardiac electrogram signal in which left bundle branch tissue features can be detected during rotation of the implantable medical lead.

13. The system of claim 11, wherein the adjustable rotational connector is configured to conduct a cardiac electrogram signal in which left bundle branch tissue features can be detected during rotation of the implantable medical lead.

14. The system of claim 11, wherein the adjustable rotational connector is configured to conduct a cardiac electrogram signal in which His-Purkinje conduction system features can be detected during rotation of the implantable medical lead.

15. The system of claim 11, further comprising an electrical contact structure configured to establish electrical communication between the pin and the cable, wherein the pin is rotatable along a longitudinal axis relative to the contact surface, and wherein the pin comprises:
a pin base defining a recess;
a pin tip; and
a pin biasing member configured to force the pin tip against the contact surface.

16. The system of claim 11, wherein the adjustable socket is configured to receive a lead connector of an IS-1 connector, a DF-4 connector, and an IS-4 lead connector size configuration.

17. The system of claim 11, wherein the actuatable element defines a channel in which the adjustable socket and an elastically deformable member are at least partially positioned, wherein the elastically deformable member is configured to provide a biasing force to move the actuatable element to the second position, and wherein a portion of the channel is dimensioned such that a wall defining the portion is engaged to the adjustable socket to radially compress the adjustable socket onto the lead connector while the actuatable element is in the second position relative to the adjustable socket.

18. The system of claim 11, wherein at least a section of the portion has an inner diameter that is less than an inner diameter of the channel immediately distal of the section such that the wall is disengaged from the adjustable socket while the actuatable element is in the first position relative to the adjustable socket.

19. The system of claim 11, wherein the adjustable socket defines define one or more indentations that allows the socket to elastically deform to receive the lead connector.

20. A method comprising:
surrounding, by an actuatable element, at least a portion of an adjustable socket of an adjustable rotational connector, wherein the adjustable rotational connector is configured to establish electrical communication between an implantable medical lead and a cable of an external testing device, wherein the adjustable socket is electrically conductive and configured to elastically deform to receive a lead connector of the implantable medical lead, wherein a proximal end of the socket is mechanically and electrically coupled to a pin of the adjustable rotational connector to provide electrical communication between the pin and the lead connector, and wherein the adjustable socket is configured to receive lead connectors of different sizes;
while transitioning from a second position relative to the adjustable socket to a first position relative to the adjustable socket, mechanically disengaging, by the actuatable element, the adjustable socket to allow the adjustable socket to elastically deform to receive the lead connector of the implantable medical lead; and
while transitioning from the second position relative to the adjustable socket to the first position relative to the adjustable socket, mechanically engaging, by the actuatable element, the adjustable socket to secure the lead connector of the implantable medical lead in the socket; and rotating, by a bearing of the adjustable rotational connector, the pin relative to the cable.
